(19) United States
(12) Patent Application Publication
(10) Pub. No.: US 2026/0014291 A1
(43) Pub. Date: Jan. 15, 2026

(54) DEVICE FOR EVAPORATING VOLATILE SUBSTANCES

(71) Applicant: ZOBELE HOLDING SPA, TRENTO (IT)

(72) Inventors: Alberto Jose SANCHO MARZO, BARCELONA (ES); Carla GARCIA NEILA, BARCELONA (ES); Jesus Orlando RAMIREZ ALVARADO, BARCELONA (ES)

(21) Appl. No.: 18/994,819

(22) PCT Filed: Jul. 13, 2023
(86) PCT No.: PCT/EP2023/069494

§ 371 Date: Jan. 15, 2025

(30) Foreign Application Priority Data
Jul. 15, 2022 (ES) P202230653

(51) Int. Cl. A61L 9/12

(52) U.S. Cl. CPC A61L 9/127; A61L 2209/133; A61L 2209/15

(57) ABSTRACT

The evaporation device comprises a container (1) in which the volatile substances are contained; a wick (3), which absorbs the volatile substances, through which the volatile substances evaporate; and a stopper (2) which is attachable to said container (1) and can be placed in a closed position, in which the evaporation of the volatile substances is prevented, and an evaporation position, in which the wick (3) is exposed to the environment and the evaporation of the volatile substances is allowed, and also comprises a clamping element (4), movable between a clamping position, in which the clamping element (4) clamps the wick (3), and a release position, in which the clamping element (4) is separated from the wick (3). It allows to provide a device for evaporating volatile substances that allows to remove the wick and replace it with a new one in a simple way.

### OBJECT OF THE INVENTION

The present invention relates to a device for evaporating volatile substances, which allows the removal of a wick from the device and its replacement with a new wick.

### BACKGROUND OF THE INVENTION

The use of volatile substance refills in volatile substance evaporation devices is well known.

These refills consist of a container containing a liquid in which a porous element or wick is placed. The upper part of the wick extends out of the container, and it is this upper part where the evaporation of the volatile substances takes place, either passively, without any other means to enhance evaporation, or under the action of a heater, fan, nebulizer or any known means of diffusion.

Devices for the evaporation of volatile substances without energy supply, i.e., without batteries or connection to a power supply, are not designed to be reusable, but rather the opposite.

Nowadays, such evaporation devices for volatile substances are designed to prevent users from being able to remove the wick, to prevent the user from refilling the container with any substance that has not been validated for that use, and also for safety reasons, to prevent anyone from drinking the volatile substance once the wick has been removed.

In addition, if the user wants to do so, removing the wick is difficult and messy. The consumer may need tools and the risk of getting his hands dirty with oil containing volatile substances is high.

### DESCRIPTION OF THE INVENTION

Therefore, an objective of the present invention is to provide a device for evaporating volatile substances which allows the wick to be removed and replaced by a new one in a simple manner.

The device for evaporating volatile substances according to the present invention is defined in claim 1. Optional additional features are included in the dependent claims.

In particular, the device for evaporating volatile substances comprises:
a container in which volatile substances are housed;
a wick, which absorbs the volatile substances, through which the volatile substances evaporate; and
a stopper attachable to the container, which can be placed in a closed position, where the evaporation of the volatile substances is prevented, and an evaporation position, where the wick is exposed to the environment and the evaporation of the volatile substances is allowed, and
a clamping element, movable between a clamping position, in which the clamping element clamps the wick, and a release position, in which the clamping element is separated from the wick.

According to a preferred embodiment, the clamping element comprises a central body and two elastic arms, each elastic arm comprising a gripping element on its side furthest from the central body, so that pressure on the central body causes the gripping elements to move towards each other.

Preferably, the elastic arms of the clamping element are curved, and the clamping element also comprises a connecting portion between the elastic arms.

According to a preferred embodiment, said connecting portion is elastic and U-shaped.

In addition, the ends of the connecting portion are preferably attached to the ends of the elastic arms furthest away from the central body.

The stopper of the device according to the present invention preferably comprises a central hole, where the clamping element is housed, and the central body of the clamping element is at the same level as the top of the stopper in its release position.

The container preferably comprises a neck, the gripping elements of the clamping element being in contact with the neck in its release position.

The stopper further comprises an inner body, which is located between the wick and the clamping element, the inner body of the stopper being preferably cylindrical in shape.

With the device for evaporating volatile substances according to the present invention it is possible that the different components of the device are properly recycled, e.g., the glass bottle, the wick, the stopper, etc.

In addition, it allows the reuse of part of the components to assemble a new refill, i.e., a new wick and refill the container.

It also ensures the integrity of the wick when it is in use but allows it to be easily and cleanly separated when the wick life is over.

### DESCRIPTION OF THE DRAWINGS

In order to complement the description being made and in order to assist in a better understanding of the features of the invention, in accordance with a preferred example of a practical embodiment thereof, a set of drawings is attached hereto as an integral part of the said description, in which the following is illustratively and non-limitingly depicted:

FIG. 1 is an exploded perspective view of the device for evaporating volatile substances according to the present invention;

FIG. 2 is a perspective section view of the device for evaporating volatile substances according to the present invention with its stopper in its closed position and the clamping element in its release position;

FIG. 3 is a perspective section view of the device for evaporating volatile substances according to the present invention with its stopper in its evaporation position and the clamping element in its clamping position; and

FIG. 4 is a perspective section view of the stopper of the device for evaporating volatile substances according to the present invention with the wick attached, removed from the rest of the evaporation device for volatile substances.

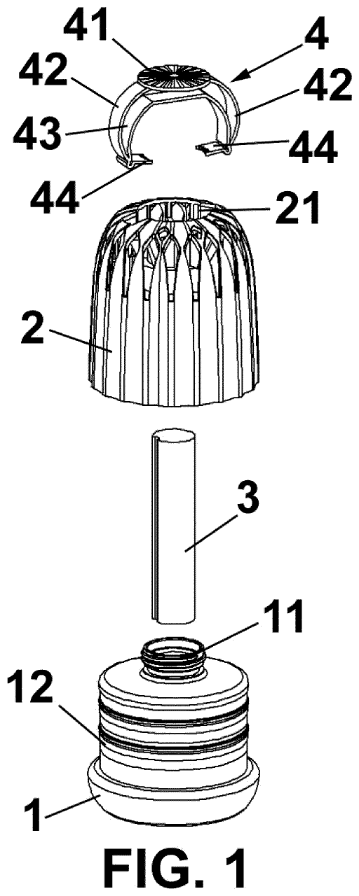

### PREFERRED EMBODIMENT OF THE INVENTION

The device for evaporating volatile substances according to a present invention comprises a container (1) in which the volatile substances are housed. This container (1) is, for example, made of glass, and contains within it a liquid containing the volatile substances.

Furthermore, according to the embodiment shown, the container (1) comprises a neck (11) and may comprise threads (12) or ribs for the removable and/or displaceable attachment of a stopper (2).

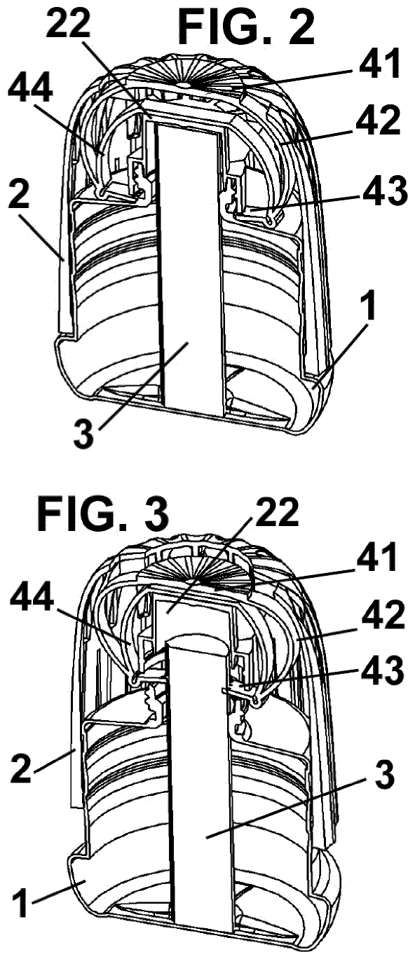

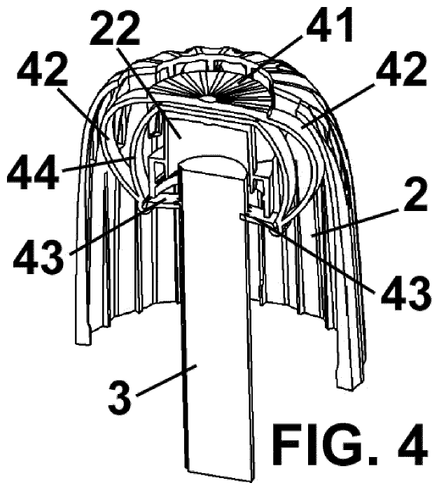

Said stopper (2) is attachable to said container (1), and can be placed in a closing position, shown in FIG. 2, in which evaporation of the volatile substances is prevented, and an evaporation position, shown in FIG. 3, in which a wick (3) is exposed to the environment and evaporation of the volatile substances is allowed.

The wick (3) is partially placed inside the container (1) to absorb the volatile substances contained in the container (1), and a part of the wick (3) is placed outside the container (1), through which the volatile substances evaporate into the environment. The wick (3) is made of any porous material suitable to allow the absorption and evaporation of the volatile substances.

The device for evaporating volatile substances also comprises a clamping element (4), which is movable between a clamping position, shown in FIG. 3, in which the clamping element (4) clamps the wick (3), and a release position, in which the clamping element (4) is detached from the wick (3).

According to the embodiment shown, the clamping element (4) comprises a central body (41) and two elastic arms (42), each elastic arm (42) comprising a gripping element (43) on its side furthest from the central body (41), so that pressure on the central body (41) causes the gripping elements (43) to move towards each other.

The central body (41) is preferably disc-shaped, and the elastic arms (42) of the clamping element (4) are curved.

Furthermore, the clamping element (4) also comprises a connecting portion (44) between the elastic arms (42), which is also elastic, and preferably U-shaped. The ends of said connecting portion (44), according to the embodiment shown, are attached to the ends of the elastic arms (42) furthest away from the central body (41).

Said clamping element (4) is located inside the stopper (2), in a hole (21), preferably circular, located in a position centered on the top of the stopper (2). As can be seen in FIG. 2, in the closed position of the stopper (2), the central body (41) of the clamping element (4) is flush or at the same level as the top of the stopper (2).

The stopper (2) also comprises an inner body (22), which is located between the wick (3) and the clamping element (4), as can be seen in FIGS. 2 and 3. The inner body (22) of the stopper (2) is preferably cylindrical in shape.

The device for evaporating volatile substances according to the present invention has three possible positions.

The first position is for the transport of the device according to the present invention. In this position, the container (1) is closed by the stopper (3), which is in its closed position, shown in FIG. 2, fulfilling the function of preventing leakage of the liquid contained inside the container (1) during transport, and the clamping element (4) is in its released position, i.e. it does not contact the wick (3), for example, it may be in contact with the neck (11) of the container (1).

The second position is the evaporation position. For activation, the stopper (2) is moved in relation to the container (1), i.e., it is raised vertically according to the arrangement shown in the figures. In this way, the wick (3) is exposed to the air stream and carries away the volatile substances so that the product is activated.

When the product runs out, the user has the option of removing the wick (3) by activating the clamping element (4). To do this, the central body (41) must be pressed as a button. This movement is transmitted by the elastic arms (42) and/or the connection portion (44), causing the gripping elements (43) to move towards each other, and forces the grip on the wick (3) by these gripping elements (43), which preferably comprise teeth.

Once the clamping element (4) is attached to the wick (3), the plug (2), together with the clamping element (4) and the wick (3), is completely removed. To do this, the user holds down the central body (41) of the clamping element (4).

The clamping element (4) holds the wick (3) for as long as the user presses the central body (41), allowing it to be transported, for example, to a recycling container. Once the user decides that it is time to release the wick (3), the user stops pressing the central body (41) and the wick (3) is released and falls, for recycling, without the need to touch it with the hands.

The device according to the present invention is then ready to be fitted with a new wick for reuse.

In this way, it is possible to recycle the wick (3) in a controlled manner, by means of a pressing and releasing movement once the pressure is released, without contaminating the hands with the substance contained in the container (1), in a clean and safe manner for the user.

Although reference has been made to a specific embodiment of the invention, it is obvious to a person skilled in the art that the device for evaporating volatile substances described is susceptible to numerous variations and modifications, and that all the details mentioned can be replaced by technically equivalent ones, without departing from the scope of protection defined by the appended claims.

1. A device for evaporating volatile substances, comprising:
a container (1) in which the volatile substances are contained;
a wick (3), which absorbs volatile substances, through which the volatile substances evaporate; and
a stopper (2) which is attachable to the container (1) and can be placed in a closed position, in which the evaporation of the volatile substances is prevented, and an evaporation position, in which the wick (3) is exposed to the environment and the evaporation of the volatile substances is allowed,
wherein the device for evaporating volatile substances also comprises a clamping element (4), movable between a clamping position, in which the clamping element (4) clamps the wick (3), and a release position, in which the clamping element (4) is separated from the wick (3).

2. Device for evaporating volatile substances according to claim 1, wherein the clamping element (4) comprises a central body (41) and two elastic arms (42), each elastic arm (42) comprising a gripping element (44) on its side furthest from the central body (41), so that pressure on the central body (41) causes the gripping elements (44) to move towards each other.

3. Device for evaporating volatile substances according to claim 2, wherein the elastic arms (42) of the clamping element (4) are curved.

4. Device for evaporating volatile substances according to claim 2, wherein the clamping element (4) also comprises a connecting portion (44) between the elastic arms (42).

5. Device for evaporating volatile substances according to claim 4, wherein said connecting portion (44) is U-shaped.

6. Device for evaporating volatile substances according to claim 4, wherein said connecting portion (44) is elastic.

7. Device for evaporating volatile substances according to claim 4, wherein the ends of the connecting portion (44) are attached to the ends of the elastic arms (42) further away from the central body (41).

8. Device for evaporating volatile substances according to claim 1, wherein the stopper (2) comprises a central hole (21), in which the clamping element (4) is housed.

9. Device for evaporating volatile substances according to claim 8, wherein the central body (41) of the clamping element (4) is at the same level as the upper part of the stopper (2) in its release position.

10. Device for evaporating volatile substances according to claim 1, wherein the container (1) comprises a neck (11), the gripping elements (43) of the clamping element (4) being in contact with said neck (11) in their release position.

11. Device for evaporating volatile substances according to claim 1, wherein the stopper (2) comprises an inner body (22), which is located between the wick (3) and the clamping element (4).

12. Device for evaporating volatile substances according to claim 11, wherein the inner body (22) of the stopper (2) is cylindrical in shape.
